(19) United States
(12) Patent Application Publication
(10) Pub. No.: US 2025/0034113 A1
(43) Pub. Date: Jan. 30, 2025

(54) SOLID STATE FORMS OF TAVAPADON AND PROCESSES FOR PREPARATION THEREOF

(71) Applicant: TEVA CZECH INDUSTRIES S.R.O, Opava-Komarov (CZ)

(72) Inventors: Zuzana Przeczkova, Ostrava (CZ); Adéla Bártová, Ostrava-Michalkovice (CZ); Pavel Kolesa, Hlubocec (CZ); Alexandr Jegorov, Dobra Voda (CZ); Jana Romanová, Hrcava (CZ)

(21) Appl. No.: 18/714,646

(22) PCT Filed: Dec. 1, 2022
(86) PCT No.: PCT/US2022/051470

§ 371 Date: May 30, 2024

Related U.S. Application Data
(60) Provisional application No. 63/309,726, filed on Feb. 14, 2022.
(60) Provisional application No. 63/285,566, filed on Dec. 3, 2021.

(51) Int. Cl. C07D 401/12; A61K 31/513

(52) U.S. Cl. CPC C07D 401/12; A61K 31/513

(57) ABSTRACT

The present disclosure encompasses solid state forms of Tavapadon, processes for preparation thereof, and pharmaceutical compositions thereof.

### FIELD OF THE DISCLOSURE

The present disclosure encompasses solid state forms of Tavapadon, processes for preparation thereof, and pharmaceutical compositions thereof.

### BACKGROUND OF THE DISCLOSURE

Tavapadon, 1,5-dimethyl-6-(2-methyl-4-{[3-(trifluoromethyl)pyridin-2-yl]oxy}phenyl)pyrimidine-2,4(1H,3H)-dione has the following chemical structure:

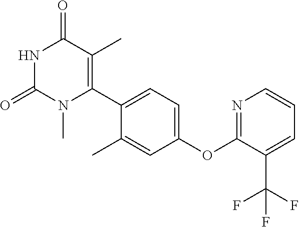 [SMILES: Cc1cc(Oc2ncccc2C(F)(F)F)ccc1-c1c(C)c(=O)[nH]c(=O)n1C]

and is described in International Publication No. WO2014/207601. As disclosed in International Publication No. WO2014/207601, the class of compounds encompassing Tavapadon, may exist as conformational isomers due to hindered rotation about a single bond, i.e., atropisomerism. In the case of Tavapadon, the atropenantiomers or atropisomers may be designated as (−)-Tavapadon or (+)-Tavapadon, depending on the optical rotation, and can include any mixture thereof, including a racemic mixture, wherein the racemic mixture can be designated as (±)-Tavapadon. According to International Publication No. WO2014/207601, the compound Tavapadon may be obtained as a racemate (i.e., (±)-Tavapadon), which may be separated by chiral chromatography into two atropenantiomers. The atropisomer exhibiting an anticlockwise (negative) rotation on a polarimeter is denoted the (−)-atropenantiomer [i.e., (−)-Tavapadon], and the atropisomer exhibiting a clockwise (positive) rotation on a polarimeter is denoted the (+)-atropenantiomer [i.e., (+)-Tavapadon].

Tavapadon belongs to the group of D1 Modulators (e.g., D1 agonist or partially agonist) which are useful in treating various D1-associated disorders such as treatment for Parkinson's disease.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for finding solid state forms (including solvated forms) of Tavapadon.

### SUMMARY OF THE DISCLOSURE

The present disclosure provides crystalline polymorphs of Tavapadon, as well as co-crystal and/or salts of Tavapadon; processes for preparation thereof, and pharmaceutical compositions thereof. These crystalline polymorphs and crystalline salts and co-crystals can be used to prepare other solid state forms of Tavapadon, Tavapadon co-crystals, Tavapadon salts and their solid state forms.

The present disclosure also provides uses of the said solid state forms of Tavapadon, Tavapadon co-crystals and/or Tavapadon salts in the preparation of other solid state forms of Tavapadon or Tavapadon co-crystals or salts thereof.

The present disclosure also provides the said solid state forms of Tavapadon co-crystals and/or Tavapadon salts for use in the preparation of other solid state forms of Tavapadon or Tavapadon co-crystals or salts thereof.

The present disclosure provides crystalline polymorphs of Tavapadon, as well as co-crystals and/or salts of Tavapadon for use in the preparation of pharmaceutical compositions and/or formulations for use in medicine, including for the treatment of Parkinson's disease.

The present disclosure also encompasses the use of crystalline polymorphs of Tavapadon, Tavapadon co-crystals and/or Tavapadon salts of the present disclosure for the preparation of pharmaceutical compositions and/or formulations.

In another aspect, the present disclosure provides pharmaceutical compositions including crystalline polymorph of Tavapadon, Tavapadon co-crystals and/or Tavapadon salts according to the present disclosure.

In yet another embodiment, the present disclosure encompasses pharmaceutical formulations including any one or combination of the crystalline polymorphs of Tavapadon, Tavapadon co-crystals and/or Tavapadon salts or pharmaceutical compositions including the described crystalline polymorph of Tavapadon, Tavapadon co-crystals and/or Tavapadon salts and at least one pharmaceutically acceptable excipient.

The present disclosure includes processes for preparing the above mentioned pharmaceutical compositions. The processes include combining any one or a combination of the crystalline polymorphs of Tavapadon, Tavapadon co-crystals and/or Tavapadon salts with at least one pharmaceutically acceptable excipient.

The crystalline polymorphs of Tavapadon, as well as co-crystals and/or salts of Tavapadon as defined herein and the pharmaceutical compositions or formulations of the crystalline polymorphs of Tavapadon, Tavapadon co-crystals and/or Tavapadon salts may be used as medicaments, such as for the treatment of Parkinson's disease.

The present disclosure also provides methods of treating Parkinson's disease, by administering a therapeutically effective amount of any one or combination of the crystalline polymorph of Tavapadon, Tavapadon co-crystals and/or Tavapadon salts of the present disclosure, or at least one of the above pharmaceutical compositions, to a subject suffering from Parkinson's disease, or otherwise in need of the treatment.

The present disclosure also provides uses of crystalline polymorphs of Tavapadon, Tavapadon co-crystals and/or Tavapadon salts or at least one of the above pharmaceutical compositions, for the manufacture of medicaments for treating e.g., Parkinson's disease.

### BRIEF DESCRIPTION OF THE DRAWINGS

FIG. 1 shows a characteristic X-ray powder diffraction pattern (XRPD) of crystalline Form 1 of Tavapadon.

FIG. 2 shows a characteristic X-ray powder diffraction pattern (XRPD) of crystalline Form 3 of Tavapadon.

FIG. 3 shows a characteristic X-ray powder diffraction pattern (XRPD) of crystalline Form 5 of Tavapadon.

FIG. 4 shows a characteristic X-ray powder diffraction pattern (XRPD) of crystalline Form H1 of Tavapadon.

FIG. 5 shows a characteristic X-ray powder diffraction pattern (XRPD) of crystalline Form R1 of Tavapadon Racemate.

### DETAILED DESCRIPTION OF THE DISCLOSURE

The present disclosure provides crystalline polymorphs of Tavapadon as well as co-crystals and/or salts of Tavapadon; processes for preparation thereof, and pharmaceutical compositions thereof.

Solid state properties of Tavapadon and crystalline polymorphs thereof can be influenced by controlling the conditions under which Tavapadon and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Tavapadon described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Tavapadon.

In some embodiments of the disclosure, the described crystalline polymorph of Tavapadon may contain from about 1% to about 20% (w/w), from about 5% to about 20% (w/w), or from about 5% to about 10% (w/w) of one or more other crystalline polymorph of the Tavapadon.

Depending on which other crystalline polymorphs a comparison is made, the crystalline polymorph of Tavapadon of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. For example Tavapadon Form 1 and Tavapadon: 4-hydroxybenzoic acid Form H1 are particularly stable to conditions of relative humidity at elevated temperatures.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Tavapadon referred to herein as being characterized by graphical data “as depicted in” or “as substantially depicted in” a Figure will thus be understood to include any crystal form of Tavapadon characterized with the graphical data having such small variations, as are well known to the skilled person, in comparison with the Figure.

As used herein, the term “isolated” in reference to crystalline polymorph of Tavapadon of the present disclosure corresponds to a crystalline polymorph of Tavapadon that is physically separated from the reaction mixture in which it is formed. As used herein, and unless stated otherwise, the term “anhydrous” in relation to crystalline forms of Tavapadon, relates to a crystalline form of Tavapadon which does not include any crystalline water (or other solvents) in a defined, stoichiometric amount within the crystal. Moreover, an “anhydrous” form would generally: not contain more than 2%, not contain more than 1% (w/w), not more than 0.8% or not more than 0.5% of either water or organic solvents, for example determined by unit cell analysis and/or by TGA. The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state.

As used herein, crystalline Tavapadon: 4-hydroxybenzoic acid is a distinct molecular species. Crystalline Tavapadon: 4-hydroxybenzoic acid may be a co-crystal of Tavapadon and 4-hydroxybenzoic acid. Alternatively crystalline Tavapadon: 4-hydroxybenzoic acid may be a salt. Preferably, Tavapadon: 4-hydroxybenzoic acid is a co-crystal. In embodiments the molar ratio between the active pharmaceutical ingredient (Tavapadon) and the coformer (4-hydroxybenzoic acid) is between 1:2 and 2:1, preferably between 1:1 or about 1:1.

As used herein, unless stated otherwise, XRPD peaks reported herein are measured using CuKα radiation, λ=1.54187 Å, preferably at a temperature of 25±3° C.

As used herein, unless stated otherwise unit cell data are obtained using a CuKα microfocused tube (λ=1.54148 Å) at 95 K, or in the case of Form H1, at 293K.

As used herein, unless stated otherwise, chiral purity is measured by chiral HPLC, preferably on a Lux Cellulose-2 chiral column, using heptane/ethanol (1:1 v/v) with UV detection (254 nm), preferably at a temperature of 25±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

Unless otherwise indicated, the term Tavapadon generally refers to a compound having the chemical formula:

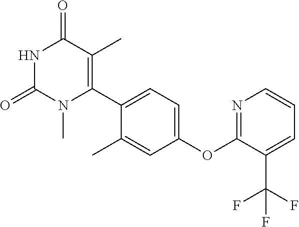 [SMILES: Cc1cc(Oc2ncccc2C(F)(F)F)ccc1-c1c(C)c(=O)[nH]c(=O)n1C]

As used herein, unless otherwise indicated, the term Tavapadon encompasses atropenantiomers or atropisomers of this compound, including (−)-Tavapadon and (+)-Tavapadon, depending on the optical rotation (i.e., negative or positive rotation, respectively as measured on a polarimeter), and can include any mixture of (−)-Tavapadon and (+)-Tavapadon in any proportion, including a racemic mixture, wherein the racemic mixture may be designated as (±)-Tavapadon. Preferably the crystalline forms of Tavapadon as described in any aspect or embodiment of the invention relate to (−)-Tavapadon.

The term (−)-Tavapadon refers to the atropisomer of Tavapadon which rotates plane polarized light in an anti-clockwise direction. As determined by single crystal absolute structure determination studies, (−)-Tavapadon may alternatively be referred to herein as Tavapadon atropisomer P (Sa). Likewise, the term (+)-Tavapadon refers to the atropisomer of Tavapadon which rotates plane polarized light in a clockwise direction, and may alternatively be referred to herein as Tavapadon atropisomer M (Ra). Particularly, the present disclosure relates to (−)-Tavapadon, i.e., Tavapadon atropisomer P (Sa).

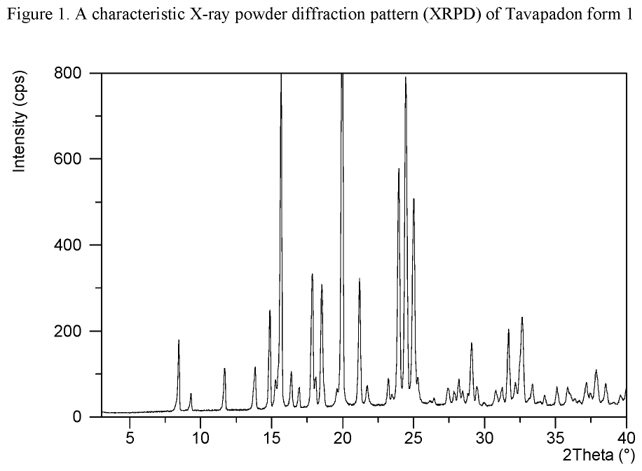

The present disclosure includes a crystalline polymorph of Tavapadon, designated Form 1. Crystalline Form 1 of Tavapadon may be characterized by data selected from one or more of the following; (a) an X-ray powder diffraction pattern substantially as depicted in FIG. 1; (b) an X-ray powder diffraction pattern having peaks at 8.5, 11.7, 14.9, 15.7 and 18.5 degrees 2-theta±0.2 degrees 2-theta; (c) unit cell parameters, measured at 95 K, substantially equal to the following:

| Unit cell parameters | Crystal structure |
|---|---|
| Cell length a | 7.482 Å |
| Cell length b | 12.521 Å |
| Cell length c | 18.885 Å |
| Cell angle alpha | 90° |
| Cell angle beta | 100.624° |
| Cell angle gamma | 90° |
| Cell volume | 1738.72 Å3 |
| Symmetry cell setting | Monoclinic |
| Symmetry space group name | P 2₁ |

or (d) combinations thereof.

Crystalline Form 1 of Tavapadon may be further characterized by an X-ray powder diffraction pattern having peaks at 8.5, 11.7, 14.9, 15.7 and 18.5 degrees 2-theta±0.2 degrees 2-theta, and also having any one, two, three, four or five additional peaks selected from 9.3, 16.9, 20.0, 24.5 and 25.0 degrees 2-theta±0.2 degrees 2-theta.

Crystalline Form 1 of Tavapadon may alternatively be characterized by an X-ray powder diffraction pattern having peaks at 8.5, 9.3, 11.7, 14.9, 15.7, 16.9, 18.5, 20.0, 24.5 and 25.0 degrees 2-theta±0.2 degrees 2-theta.

According to any aspect or embodiment of the present disclosure, crystalline Form 1 of Tavapadon is the (−)-atropisomer, i.e., (−)-Tavapadon or Tavapadon atropisomer P (Sa).

In embodiments of the present disclosure, crystalline Form 1 of Tavapadon is isolated.

Crystalline Form 1 may be polymorphically pure and/or enantiomerically pure.

Crystalline Form 1 is preferably an anhydrous form.

Crystalline Form 1 of Tavapadon may be characterized by each of the above characteristics alone or by all possible combinations, e.g., an XRPD pattern having peaks at 8.5, 11.7, 14.9, 15.7 and 18.5 degrees 2-theta±0.2 degrees 2-theta; an XRPD pattern as depicted in FIG. 1; unit cell data set forth herein; and combinations thereof.

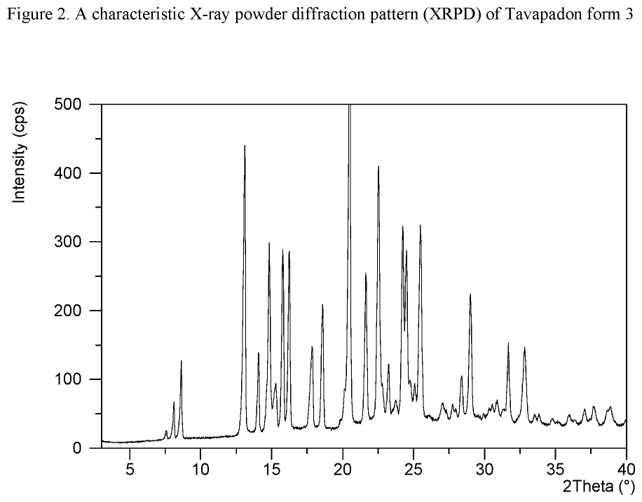

The present disclosure includes a crystalline polymorph of Tavapadon, designated Form 3. Crystalline Form 3 of Tavapadon may be characterized by data selected from one or more of the following: an X-ray powder diffraction pattern substantially as depicted in FIG. 2; an X-ray powder diffraction pattern having peaks at 8.1, 8.6, 13.1, 14.1 and 15.8 degrees 2-theta±0.2 degrees 2-theta; and combinations of these data.

Crystalline Form 3 of Tavapadon may be further characterized by an X-ray powder diffraction pattern having peaks at 8.1, 8.6, 13.1, 14.1 and 15.8 degrees 2-theta±0.2 degrees 2-theta, and also having any one, two, three, four or five additional peaks selected from 16.3, 18.6, 20.5, 21.6 and 22.5 degrees 2-theta±0.2 degrees 2-theta.

According to any aspect or embodiment of the present disclosure, crystalline Form 3 of Tavapadon is the (−)-atropisomer, i.e., (−)-Tavapadon, i.e., (−)-Tavapadon or Tavapadon atropisomer P (Sa).

In embodiments of the present disclosure, crystalline Form 3 of Tavapadon is isolated.

Crystalline Form 3 of Tavapadon may be a solvate. In embodiments, crystalline Form 3 is an anisole solvate.

Crystalline Form 3 may be polymorphically pure and/or enantiomerically pure.

Crystalline Form 3 of Tavapadon may be characterized by each of the above characteristics alone or by all possible combinations, e.g., an XRPD pattern having peaks at 8.1, 8.6, 13.1, 14.1 and 15.8 degrees 2-theta±0.2 degrees 2-theta; an XRPD pattern as depicted in FIG. 2; and combinations thereof.

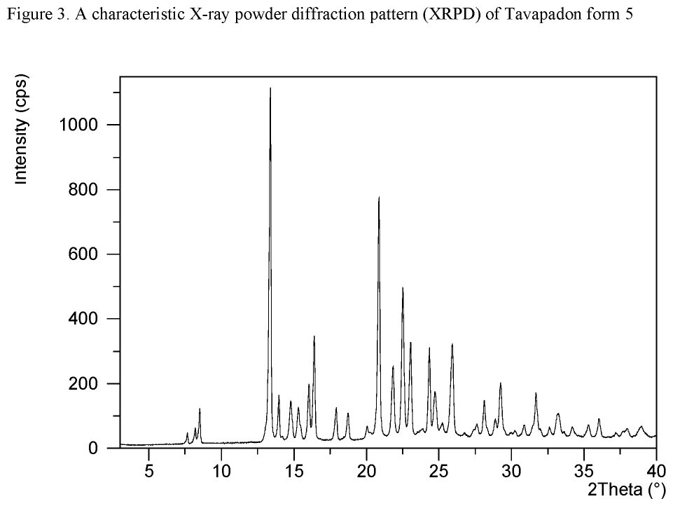

The present disclosure further includes a crystalline polymorph of Tavapadon, designated Form 5. Crystalline Form 5 of Tavapadon may be characterized by data selected from one or more of the following: an X-ray powder diffraction pattern substantially as depicted in FIG. 3; an X-ray powder diffraction pattern having peaks at 8.5, 13.4, 16.4, 20.9 and 22.5 degrees 2-theta±0.2 degrees 2-theta; and combinations of these data.

Crystalline Form 5 of Tavapadon may be further characterized by an X-ray powder diffraction pattern having peaks at 8.5, 13.4, 16.4, 20.9 and 22.5 degrees 2-theta±0.2 degrees 2-theta, and also having any one, two, three, four or five additional peaks selected from 7.7, 14.0, 14.8, 21.8 and 23.1 degrees 2-theta±0.2 degrees 2-theta.

According to any aspect or embodiment of the present disclosure, crystalline Form 5 of Tavapadon is the (−)-atropisomer, i.e., (−)-Tavapadon, i.e., (−)-Tavapadon or Tavapadon atropisomer P (Sa).

In embodiments of the present disclosure, crystalline Form 5 of Tavapadon is isolated.

Crystalline Form 5 of Tavapadon may be a solvate. In embodiments, crystalline Form 5 is a toluene solvate. In embodiments the molar ratio between Tavapadon and Toluene is between 2:1 to 1:2, preferably 2:1

Crystalline Form 5 may be polymorphically pure and/or enantiomerically pure.

Crystalline Form 5 of Tavapadon may be characterized by each of the above characteristics alone or by all possible combinations, e.g., an XRPD pattern having peaks at 8.5, 13.4, 16.4, 20.9 and 22.5 degrees 2-theta±0.2 degrees 2-theta; an XRPD pattern as depicted in FIG. 3; and combinations thereof.

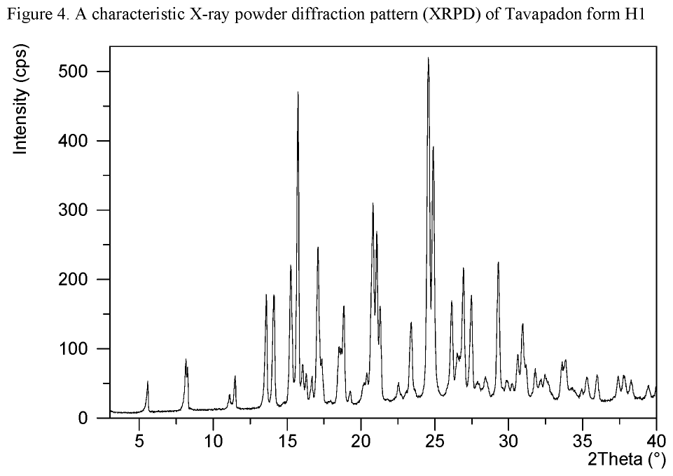

The disclosure further encompasses a crystalline form of Tavapadon: 4-hydroxybenzoic acid, designated form H1. Crystalline Form H1 of Tavapadon: 4-hydroxybenzoic acid may be characterized by data selected from one or more of the following: (a) an X-ray powder diffraction pattern substantially as depicted in FIG. 4; (b) an X-ray powder diffraction pattern having peaks at 5.6, 13.6, 14.1, 15.3 and 15.8 degrees 2-theta±0.2 degrees 2-theta; (c) unit cell parameters, measured at 293 K, substantially equal to the following:

| Unit cell parameters | Crystal structure |
|---|---|
| Cell length a | 17.0584 Å |
| Cell length b | 6.8352 Å |
| Cell length c | 22.9267 Å |
| Cell angle alpha | 90° |
| Cell angle beta | 110.9631° |
| Cell angle gamma | 90° |
| Cell volume | 1738.72(3) Å3 |
| Symmetry cell setting | monoclinic |
| Symmetry space group name | P 2₁/c |

and (d) combinations of these data.

Crystalline Form H1 of Tavapadon: 4-hydroxybenzoic acid may be further characterized by an X-ray powder diffraction pattern having peaks at 5.6, 13.6, 14.1, 15.3 and 15.8 degrees 2-theta±0.2 degrees 2-theta, and also having any one, two, three, four or five additional peaks selected from 11.5, 23.4, 24.6, 26.2 and 29.3 degrees 2-theta±0.2 degrees 2-theta. According to any aspect or embodiment of the present disclosure, crystalline Form H1 of Tavapadon: 4-hydroxybenzoic acid is a co-crystal, preferably wherein the molar ratio between Tavapadon and 4-hydroxybenzoic acid is between 1:2 and 2:1. More preferably, crystalline Form H1 of Tavapadon: 4-hydroxybenzoic acid is a co-crystal having a ratio of Tavapadon:4-hydroxybenzoic acid of about 1:1.

According to any aspect or embodiment of the present disclosure, crystalline Form H1 of Tavapadon: 4-hydroxybenzoic acid is the (−)-atropisomer, i.e., (−)-Tavapadon, i.e., (−)-Tavapadon or Tavapadon atropisomer P (Sa).

In embodiments of the present disclosure, crystalline Form H1 of Tavapadon: 4-hydroxybenzoic acid is isolated.

Crystalline form H1 of Tavapadon: 4-hydroxybenzoic acid may be polymorphically pure and/or enantiomerically pure.

Crystalline Form H1 of Tavapadon: 4-hydroxybenzoic acid may be characterized by each of the above characteristics alone or by all possible combinations, e.g., an XRPD pattern having peaks at 5.6, 13.6, 14.1, 15.3 and 15.8 degrees 2-theta±0.2 degrees 2-theta; an XRPD pattern as depicted in FIG. 4; and combinations thereof.

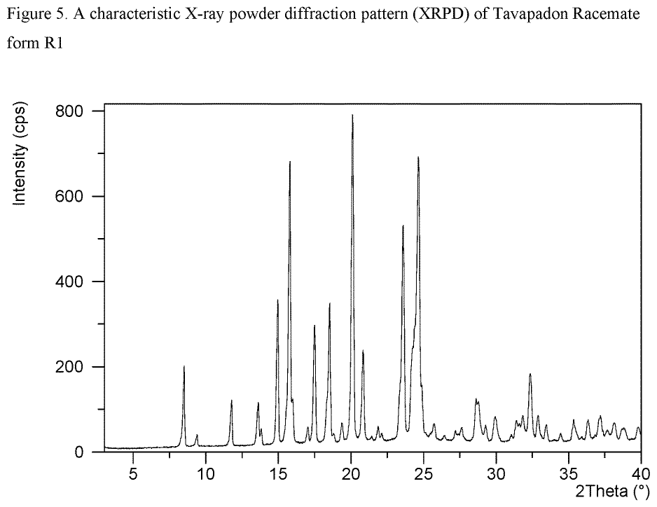

The present disclosure further includes a crystalline polymorph of Tavapadon, designated Form R1. Crystalline Form R1 of Tavapadon may be characterized by data selected from one or more of the following: an X-ray powder diffraction pattern substantially as depicted in FIG. 5; an X-ray powder diffraction pattern having peaks at 8.5, 11.8, 15.0, 18.5 and 20.1 degrees 2-theta±0.2 degrees 2-theta; and combinations of these data.

Crystalline Form R1 of Tavapadon may be further characterized by an X-ray powder diffraction pattern having peaks at 8.5, 11.8, 15.0, 18.5 and 20.1 degrees 2-theta 0.2 degrees 2-theta, and also having any one, two, three, four or five additional peaks selected from 9.4, 15.8, 17.5, 20.8 and 23.6 degrees 2-theta±0.2 degrees 2-theta.

According to any aspect or embodiment of the present disclosure, crystalline Form R1 of Tavapadon is a racemic mixture designated as (±)-Tavapadon

In embodiments of the present disclosure, crystalline Form R1 of Tavapadon is isolated.

Crystalline Form R1 of Tavapadon may be an anhydrous form.

Crystalline Form R1 may be polymorphically pure.

Crystalline Form R1 of Tavapadon may be characterized by each of the above characteristics alone or by all possible combinations, e.g., an XRPD pattern having peaks at 8.5, 11.8, 15.0, 18.5 and 20.1 degrees 2-theta±0.2 degrees 2-theta; an XRPD pattern as depicted in FIG. 5; and combinations thereof.

In any aspect or embodiment of the present disclosure, any of the solid state forms of Tavapadon described herein may be polymorphically pure or may be substantially free of any other solid state forms of the subject Tavapadon or Tavapadon solvates (for example crystalline Form 1 of Tavapadon, which is polymorphically pure, may be substantially free of any other solid state forms of Tavapadon; and crystalline Form 3 of Tavapadon which is polymorphically pure, may be substantially free of any other solid state forms of Tavapadon). In any aspect or embodiment of the present disclosure, any of the solid state forms of Tavapadon described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound (i.e., Tavapadon), preferably as measured by XRPD. Thus, any of the disclosed crystalline forms of Tavapadon, described herein may be substantially free of any other solid state forms of the subject Tavapadon, respectively, and may contain greater than about 80% (w/w), greater than about 90% (w/w), greater than about 95% (w/w), greater than about 98% (w/w), greater than about 99% (w/w), or about 100% of the subject solid state form of Tavapadon.

In any aspect or embodiment of the present disclosure, any of the solid state forms of Tavapadon described herein may be enantiomerically pure or may be substantially free of any enantiomeric forms of the subject Tavapadon. In particular, in any aspect or embodiment of the present disclosure, any of the solid state forms of Tavapadon may be enantiomerically pure or substantially free of other enantiomers of Tavapadon, i.e., in any aspect or embodiment of the present invention, the solid state form of Tavapadon may be atropisomerically pure, or substantially free of other atropisomers of Tavapadon. Thus, a solid state form of (−)-Tavapadon according to any aspect or embodiment of the disclosure may be atropisomerically pure, or substantially free of (+)-Tavapadon. Accordingly, Form 1 of (−)-Tavapadon which is polymorphically pure, may be substantially free of (+)-Tavapadon; and crystalline Form 3 of Tavapadon which is polymorphically pure, may be substantially free of (+)-Tavapadon. In any aspect or embodiment of the present disclosure, any of the solid state forms of a Tavapadon atropisomer, particularly any of the solid state forms of (−)-Tavapadon, described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of other Tavapadon atropisomers, particularly (+)-Tavapadon, preferably as measured by chiral HPLC. Thus, any of the disclosed crystalline forms of Tavapadon atropisomer, particularly (−)-Tavapadon, described herein may be substantially free of other atropisomers, particularly (+)-Tavapadon, and preferably may contain greater than about 80% (w/w), greater than about 90% (w/w), greater than about 95% (w/w), greater than about 98% (w/w), greater than about 99% (w/w), greater than about 99.5% (w/w), greater than about 99.8% (w/w), greater than about 99.9% (w/w), or about 100% of the (−)-atropisomer of Tavapadon.

The present disclosure also provides the use of the solid state forms of Tavapadon of the present disclosure for preparing different solid state forms of Tavapadon.

The present disclosure further encompasses processes for preparing different solid state forms of Tavapadon. The process includes preparing at least one of the solid state forms of Tavapadon of the present disclosure, and converting it to different solid state forms of Tavapadon, or Tavapadon salts or co crystal thereof.

The present disclosure may further comprise a step of combining the crystalline forms with at least one pharmaceutically acceptable excipient to provide a pharmaceutical composition.

The present disclosure provides the above described crystalline polymorph of Tavapadon for use in the preparation of pharmaceutical compositions comprising Tavapadon, Tavapadon salts or co-crystals and/or crystalline polymorphs thereof.

The present disclosure also encompasses the use of crystalline polymorphs of the present disclosure for the preparation of pharmaceutical compositions of crystalline polymorph Tavapadon, Tavapadon co-crystals or salts and/or crystalline polymorphs thereof.

The present disclosure includes processes for preparing the above mentioned pharmaceutical compositions. The processes include combining the crystalline polymorphs of Tavapadon of the present disclosure with at least one pharmaceutically acceptable excipient.

Pharmaceutical combinations or formulations of the present disclosure contain the solid state forms of Tavapadon of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Tavapadon can be administered. Tavapadon forms of the present disclosure may be formulated for administration to a mammal, in embodiments to a human. Tavapadon can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Tavapadon and the pharmaceutical compositions and/or formulations of Tavapadon of the present disclosure can be used as medicaments, in embodiments in the treatment of Parkinson's disease.

The present disclosure also provides methods of treating Parkinson's disease by administering a therapeutically effective amount of the crystalline forms of Tavapadon or at least one of the above pharmaceutical compositions and/or formulations, to a subject in need of the treatment.

Further aspects and embodiments of the present disclosure are set out in the numbered clauses below:
1. A crystalline Form 1 of Tavapadon, which is characterized as having at least one of:
   a. an XRPD pattern having peaks at 8.5, 11.7, 14.9, 15.7 and 18.5 degrees 2-theta±0.2 degrees 2-theta;
   b. an XRPD pattern as depicted in FIG. 1;
   c. unit cell parameters substantially equal to the following, when measured at 95 K:

| Unit cell parameters | Crystal structure |
|---|---|
| Cell length a | 7.482 Å |
| Cell length b | 12.521 Å |
| Cell length c | 18.885 Å |
| Cell angle alpha | 90° |
| Cell angle beta | 100.624° |
| Cell angle gamma | 90° |
| Cell volume | 1738.72 Å3 |
| Symmetry cell setting | Monoclinic |
| Symmetry space group name | P 2₁ |

2. A crystalline product according to Clause 1, which is characterized by a) and c), or which is characterized by b) and c).
3. A crystalline product according to Clause 1 or Clause 2, characterized by the XRPD pattern having peaks at 8.5, 11.7, 14.9, 15.7 and 18.5 degrees 2-theta±0.2 degrees 2-theta, and also having one, two, three or four additional peaks selected from 9.3, 16.9, 20.0, 24.5 and 25.0 degrees 2-theta±0.2 degrees 2-theta.
4. Crystalline Form 1 of Tavapadon characterized by an X-ray powder diffraction pattern having peaks at 8.5, 9.3, 11.7, 14.9, 15.7, 16.9, 18.5, 20.0, 24.5 and 25.0 degrees 2-theta±0.2 degrees 2-theta.
5. Crystalline Form 3 of Tavapadon, which is characterized by data selected from:
   a. an X-ray powder diffraction pattern having peaks at 8.1, 8.6, 13.1, 14.1 and 15.8 degrees 2-theta±0.2 degrees 2-theta; or
   b. an X-ray powder diffraction pattern substantially as depicted in FIG. 2.
6. Crystalline Form 3 of Tavapadon according to Clause 5, which is characterized by an X-ray powder diffraction pattern having peaks at 8.1, 8.6, 13.1, 14.1 and 15.8 degrees 2-theta±0.2 degrees 2-theta, and also having any one, two, three, four or five additional peaks selected from 16.3, 18.6, 20.5, 21.6 and 22.5 degrees 2-theta±0.2 degrees 2-theta.
7. A crystalline product according any one or more of Clauses 1, 2, 3, 4, 5, and 6, which is isolated.
8. A crystalline product according to any one or more of Clauses 1, 2, 3, 4, 5, 6, and 7, which is polymorphically pure.
9. A crystalline product according to any one or more of Clauses 1, 2, 3, 4, 5, 6, 7, and 8, which contains: no more than about 20%, no more than about 10%, no more than about 5%, no more than about 2%, no more than about 1% or about 0% of any other crystalline forms of Tavapadon.
10. A crystalline product according to any one or more of Clauses 1, 2, 3, 4, 5, 6, 7, 8, and 9, which contains: no more than about 20%, no more than about 10%, no more than about 5%, no more than about 2%, no more than about 1% or about 0% of amorphous Tavapadon.
11. A crystalline product according to any one or more of Clauses 1, 2, 3, 4, 5, 6, 7, 8, 9, and 10, which is enantiomerically pure.
12. A crystalline product according to any one or more of Clauses 1, 2, 3, 4, 5, 6, 7, 8, 9, 10, and 11, which is atropisomerically pure.
13. A crystalline product according to any one or more of Clauses 1, 2, 3, 4, 5, 6, 7, 8, 9, 10, 11, and 12, which is (−)-Tavapadon.
14. A crystalline product according to Clause 13, which is atropisomerically pure and is substantially free of (+)-Tavapadon.
15. A crystalline product according to Clause 14, which contains: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of (+)-Tavapadon.
16. A pharmaceutical composition comprising a crystalline product according to any one or more of Clauses 1, 2, 3, 4, 5, 6, 7, 8, 9, 10, 11, 12, 13, 14, and 15, and at least one pharmaceutically acceptable excipient.
17. Use of crystalline Form 1 of Tavapadon as described in any one or more of Clauses 1, 2, 3, 4, 5, 6, 7, 8, 9, 10, 11, 12, 13, 14, and 15, for the preparation of a pharmaceutical composition and/or formulation.
18. A process for preparing the pharmaceutical composition according to Clause 16, comprising combining a crystalline product according to any of Clauses 1, 2, 3, 4, 5, 6, 7, 8, 9, 10, 11, 12, 13, 14, and 15, with at least one pharmaceutically acceptable excipient.
19. Use of a crystalline product according to any one or more of Clauses 1, 2, 3, 4, 5, 6, 7, 8, 9, 10, 11, 12, 13, 14, and 15, for preparing other solid state forms of Tavapadon, Tavapadon co-crystals, Tavapadon salts and their solid state forms.
20. A crystalline product according to any of Clauses 1, 2, 3, 4, 5, 6, 7, 8, 9, 10, 11, 12, 13, 14, and 15, or a pharmaceutical composition according to Clause 16, for use as a medicament.
21. A crystalline product according to any of Claims 1, 2, 3, 4, 5, 6, 7, 8, 9, 10, 11, 12, 13, 14, and 15, or a pharmaceutical composition according to Claim 16, for use in the treatment of Parkinson's disease.
22. Crystalline Form H1 of Tavapadon: 4-hydroxybenzoic acid, which is characterized by data selected from:
   a. an X-ray powder diffraction pattern having peaks at 5.6, 13.6, 14.1, 15.3 and 15.8 degrees 2-theta±0.2 degrees 2-theta; or
   b. an X-ray powder diffraction pattern substantially as depicted in FIG. 4.
23. Crystalline Form H1 of Tavapadon: 4-hydroxybenzoic acid according to Clause 22, which is characterized by an X-ray powder diffraction pattern having peaks at 5.6, 13.6, 14.1, 15.3 and 15.8 degrees 2-theta±0.2 degrees 2-theta, and also having any one, two, three, four or five additional peaks selected from 11.5, 23.4, 24.6, 26.2 and 29.3 degrees 2-theta±0.2 degrees 2-theta.
24. A crystalline product according to any one or more of Clauses 22 and 23, which is polymorphically pure.
25. A crystalline product according to any one or more of Clauses 22, 23 and 24, which contains: no more than about 20%, no more than about 10%, no more than about 5%, no more than about 2%, no more than about 1% or about 0% of any other crystalline forms of Tavapadon.
26. A crystalline product according to any one or more of Clauses 22, 23, 24 and 25, which contains: no more than about 20%, no more than about 10%, no more than about 5%, no more than about 2%, no more than about 1% or about 0% of amorphous Tavapadon.
27. A crystalline product according to any one or more of Clauses 22, 23, 24, 25 and 26, which is enantiomerically pure.
28. A crystalline product according to any one or more of Clauses 22, 23, 24, 25, 26 and 27, which is atropisomerically pure.
29. A crystalline product according to any one or more of Clauses 22-28 which is (−)-Tavapadon.
30. A crystalline product according to Clause 29, which is atropisomerically pure and is substantially free of (+)-Tavapadon.
31. A crystalline product according to Clause 30, which contains: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of (+)-Tavapadon.
32. A pharmaceutical composition comprising a crystalline product according to any one or more of Clauses 22-31, and at least one pharmaceutically acceptable excipient.
33. Use of crystalline Form H1 of Tavapadon as described in any one or more of Clauses 22-31, for the preparation of a pharmaceutical composition and/or formulation.
34. A process for preparing the pharmaceutical composition according to Clause 32, comprising combining a crystalline product according to any of Clauses 22-31, with at least one pharmaceutically acceptable excipient.
35. Use of a crystalline product according to any one or more of Clauses 22-31, for preparing other solid state forms of Tavapadon, Tavapadon co-crystals, Tavapadon salts and their solid state forms.
36. A crystalline product according to any of Clauses 22-31, or a pharmaceutical composition according to Clause 32, for use as a medicament.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Powder X-ray Diffraction was performed on an X-Ray powder diffractometer PanAlytical X'pert Pro; CuKα radiation (λ=1.54187 Å); X'Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

### Measurement Parameters:

Scan range: 3-40 degrees 2-theta
Scan mode: continuous
Step size: 0.0167 degrees
Step size: 42 s
Sample spin: 60 rpm
Sample holder: zero background silicon plate

### Unit Cell Measurement (Form 1)

Data was collected on a Rigaku OD Supernova system equipped with Atlas S2 CCD detector and a Cu Kα microfocused tube (λ=1.54148 Å) with mirror collimation using combined φ and ω scans at 95 K. Data were corrected for absorption effects using the multi-scan method.

Data collection: CrysAlis PRO (Rigaku Oxford Diffraction, 2015); cell refinement: CrysAlis PRO; program used to solve structure: SIR92 (Altomare et al., 1994); program used to refine structure and absolute configuration analysis: CRYSTALS (Betteridge et al., 2003); Void calculation was done by Platon (Spek, 2003).

### Chiral HPLC Method

Separation of the atropisomers and measurement of the chiral purity of Tavapadon may be carried out by chiral HPLC according to the following parameters:
Column: Lux Cellulose-2 5 μm, 250×4.6 mm
Column Temperature: 23° C.
Injection Volume: 10 μl
Sample Diluent: Heptane/Dichloromethane 1:1 (v/v)
Mobile Phase: Heptane/Ethanol 1:1 (v/v)
Pump Mode: Isocratic Flow
Flow Rate: 1 ml/min
Detection: UV 254 nm, PDA 190-800 nm.

### EXAMPLES

### Preparation of Starting Materials

Tavapadon can be prepared according to methods known from the literature, for example as described in International Publication No. WO 2014/207601. For example, racemic Tavapadon may be prepared according to Examples 7 and 8 of International Publication No. WO 2014/207601, and subjected to chiral chromatography as described therein to separate the mixture into the individual atropisomers, (−)-Tavapadon and (+)-Tavapadon. In the following examples, the starting material is (−)-Tavapadon, i.e., Tavapadon atropisomer P (Sa), unless indicated otherwise

### Example 1: Preparation of Crystalline Tavapadon Form 1

Tavapadon atropisomer P (Sa) (20 mg) was dissolved in 200 μl of dichloromethane (DCM) at 40° C. The solution was then allowed to cool to room temperature spontaneously. Crystals were formed within five days. Tavapadon atropisomer P (Sa) Form 1 was obtained. The crystals were subjected to unit cell measurement as indicated above.

### Example 2: Preparation of Crystalline Tavapadon Form 1

Tavapadon atropisomer P (Sa) (5 grams) was suspended in n-propanol (62 ml) and heated to 90° C. during a period of about 45 minutes, until dissolution occurred then the clear solution was stirred at 90° C. for 30 minutes and cooled down to 20° C. during a period of about 70 minutes. Crystallization occurred at around 55° C. Next, the suspension was stirred at 20° C. for 60 minutes, filtered, washed with n-propanol and dried under vacuum for about 30 minutes. to yield Tavapadon atropisomer P(Sa) Form 1 (90%).

### Example 3: Preparation of Crystalline Tavapadon Form 3

Tavapadon atropisomer P (Sa) (1.5 grams) was dissolved in anisole (15 ml) by heating to 95° C. during a period of about 45 minutes. Clear solution was then cooled down to 20° C. during a period of about 90 minutes, material crystallized at around 32° C. Obtained suspension was stirred at 20° C. for 2 hours, filtered and dried under vacuum for 15 minutes. Solid matter (yield of 1.35 grams) was analysed by XRPD and identified as Tavapadon atropisomer P (Sa) Form 3.

### Example 4: Preparation of Crystalline Tavapadon Form 5

Tavapadon atropisomer P (Sa) (4 grams) was suspended in toluene (100 mL) and heated to 90° C. during a period of about 1 hour, the obtained clear solution was then stirred for 15 minutes at 90° C. and then cooled down to 0° C. during a period of about 90 minutes. Crystallization appeared at around 25° C. Suspension was stirred at 0° C. for 2 hours, filtered and dried under vacuum for about 15 minutes. The solid was measured by XRPD and identified as Form 5 (89% yield).

### Example 5: Preparation of Crystalline Tavapadon Form H1

### Procedure A

Tavapadon form 5 (100 mg) was suspended in 18 mL of aqueous solution of 4-hydroxybenzoic acid (3.96 mg/mL). The suspension was stirred at room temperature for about 20 hours, filtered and dried under vacuum for about 15 minutes. The solid material was measured by XRPD.

### Procedure B

Tavapadon form 5 (1 gram) was suspended in 180 mL of aqueous solution of 4-hydroxybenzoic acid (3.96 mg/mL). The suspension was stirred at room temperature for about 37 hours, filtered and dried under vacuum for about 15 minutes. The solid material was measured by XRPD.

### Procedure C

Tavapadon form 5 (4.5 grams) was suspended in 1,620 mL of aqueous solution of 4-hydroxybenzoic acid (3.96 mg/mL). The suspension was stirred at room temperature for about 5 days, filtered and dried under vacuum for about 15 minutes. The solid material was measured by XRPD.

### Procedure D

Tavapadon (2 grams) was suspended in toluene (50 mL). The suspension was heated to 130° C. to obtain clear solution. The solution was stirred for 1 minute. The solution was cooled down to room temperature and sample start to crystallize. The suspension was cooled down to 5° C. and stirred for 2 hours. Sample was filtered and dried under the vacuum (toluene solvate Form 5). Then, the toluene solvate (1 gram) was suspended in saturated aqueous solution of 4-hydroxybenzoic acid (180 mL). The suspension was stirred at room temperature for 1 day and filtrated, then the obtained sample was again suspended in saturated solution of 4-hydroxybenzoic acid (180 ml) and stirred for 39 hours. Suspension was filtered and dried under the vacuum. A sample of the crystals was ground and placed in a borosilicate-glass capillary. Powder diffraction data was collected using the Debye-Scherrer transmission configuration on the powder diffractometer Empyrean using Cu K_{α12} radiation, so λ=1.54187 Å (primary monochromator not used). The reflections positions were determined in DASH software. Indexation was done in DICVOL14 software. Hydrogens were placed in positions obtained from DFT geometry optimization.

### Example 6: Preparation of Tavapadon Racemate Form R1

Tavapadon in racemic mixture, i.e., (±)-Tavapadon (200 mg) was suspended in ethanol (10 mL) and heated to 75° C. during a period of about 30 minutes, the clear solution obtained was stirred at 75° C. for 15 minutes and cooled down to the room temperature for a period of about 90 minutes. The material was crystalized at around 45° C. Suspension was stirred at ice bath for 2 hours, filtered and dried under vacuum. The solid material was measured by XRPD.

### Example 7: Stability Test for Tavapadon Form 1 and Form H1

Samples of Tavapadon form 1 and Form H1 were subjected to conditions of different relative humidities at different temperatures. XRPD analysis was performed on the samples after 2 month. The results are shown in Table 1 below:

TABLE 1

Conditions (2 month)

| | 25° C., | 40° C., |
|---|---|---|
| | 60% RH | 75% RH |
| Tavapadon Form 1 | No change | No change |
| Tavapadon Form H1 | No change | No change |

The results demonstrate that Tavapadon form 1 and Form H1 are stable after exposure to different relative humidity conditions at different temperatures for at least 2 months, indicating that this crystalline form has good storage stability.

Samples of Tavapadon Form H1 were subjected to conditions of different relative humidities at different temperatures, over a time period of 6 months. XRPD analysis was performed on the samples after 2, 4 and 6 months. The results are shown in Table 2 below:

TABLE 2

Results
XRPD

| | | |
|---|---|---|
| Time Interval | 40° C./75% RH | 25° C./60% RH |

| | |
|---|---|
| 0 M | Form H1 |

| | | |
|---|---|---|
| 2 M | Form H1 | Form H1 |
| 4 M | Form H1 | Form H1 |
| 6 M | Form H1 | Form H1 |

The results demonstrate that Tavapadon Form H1 is stable after exposure to different relative humidity conditions at different temperatures for at least 6 months.

### Example 8: Stability Tests for Tavapadon Racemate Form R1

Samples of Tavapadon Form R1 were subjected to conditions of different relative humidities at different temperatures, over a time period of 6 months. XRPD analysis was performed on the samples after 2, 4 and 6 months. The results are shown in Table 3 below:

TABLE 3

Results
XRPD

| | | |
|---|---|---|
| Time Interval | 40° C./75% RH | 25° C./60% RH |

| | |
|---|---|
| 0 M | Form R1 |

| | | |
|---|---|---|
| 2 M | Form R1 | Form R1 |
| 4 M | Form R1 | Form R1 |
| 6 M | Form R1 | Form R1 |

The results demonstrate that Tavapadon Form R1 is stable after exposure to different relative humidity conditions at different temperatures for at least 6 months.

1. A crystalline Form 1 of Tavapadon, which is characterized as having at least one of:
a) an XRPD pattern having peaks at 8.5, 11.7, 14.9, 15.7 and 18.5 degrees 2-theta±0.2 degrees 2-theta;
b) unit cell parameters substantially equal to the following, when measured at 95 K:

| Unit cell parameters | Crystal structure |
|---|---|
| Cell length a | 7.482 Å |
| Cell length b | 12.521 Å |
| Cell length c | 18.885 Å |
| Cell angle alpha | 90° |
| Cell angle beta | 100.624° |
| Cell angle gamma | 90° |
| Cell volume | 1738.72 Å3 |
| Symmetry cell setting | Monoclinic |
| Symmetry space group name | P 2₁ |

2. The crystalline Form 1 of Tavapadon according to claim 1, which is characterized by a) and b).

3. The crystalline Form 1 of Tavapadon according to claim 1, characterized by the XRPD pattern having peaks at 8.5, 11.7, 14.9, 15.7 and 18.5 degrees 2-theta±0.2 degrees 2-theta, and also having one, two, three, four, or five additional peaks selected from 9.3, 16.9, 20.0, 24.5 and 25.0 degrees 2-theta±0.2 degrees 2-theta.

4. The crystalline Form 1 of Tavapadon according to claim 1, which is characterized by an X-ray powder diffraction pattern having peaks at 8.5, 9.3, 11.7, 14.9, 15.7, 16.9, 18.5, 20.0, 24.5 and 25.0 degrees 2-theta±0.2 degrees 2-theta.

5. The crystalline Form 1 of Tavapadon according to claim 1, which is further characterized by an XRPD pattern substantially as depicted in FIG. 1.

6. (canceled)

7. The crystalline Form 1 of Tavapadon according to claim 1, which is polymorphically pure.

8. The crystalline Form 1 of Tavapadon according to claim 1, which contains no more than about 20% of any other crystalline forms of Tavapadon.

9. The crystalline Form 1 of Tavapadon according to claim 1, which contains no more than about 20% of amorphous Tavapadon.

10. The crystalline Form 1 of Tavapadon according to claim 1, which is enantiomerically pure.

11. The crystalline Form 1 of Tavapadon according to claim 1, which is atropisomerically pure.

12. The crystalline Form 1 of Tavapadon according to claim 1, which is (−)-Tavapadon.

13. The crystalline Form 1 of Tavapadon according to claim 12, which is atropisomerically pure and is substantially free of (+)-Tavapadon.

14. The crystalline Form 1 of Tavapadon according to claim 13, which contains about 20% (w/w) or less of (+)-Tavapadon.

15-26. (canceled)

27. A pharmaceutical composition comprising the crystalline Form 1 of Tavapadon according to claim 1 and at least one pharmaceutically acceptable excipient.

28. (canceled)

29. A process for preparing a pharmaceutical composition comprising combining the crystalline Form 1 of Tavapadon according to claim 1 with at least one pharmaceutically acceptable excipient.

30. (canceled)

31. A medicament comprising the crystalline Form 1 of Tavapadon according to claim 1.

32. A method for treating Parkinson's disease, comprising administering the crystalline Form 1 of Tavapadon according to claim 1 to a patient in need of treatment.
